(19) United States
(12) Patent Application Publication
(10) Pub. No.: US 2010/0056772 A1
(43) Pub. Date: Mar. 4, 2010

(54) Method for recovering arabinogalactan (LAG) from fibrous natural plant materials

(76) Inventors: Thomas ZEILMANN, Guangzhou (CN); Niklaus Kuenzle, Ausserberg (CH); Todd Jaranson, Pengilly, MN (US)

Correspondence Address: PROPAT, L.L.C., 425-C SOUTH SHARON AMITY ROAD, CHARLOTTE, NC, 28211-2841, US

(21) Appl. No.: 12/198,960

(22) Filed: Aug. 27, 2008

(51) Int. Cl. C07H 1/00; C07H 1/08

(52) U.S. Cl. 536/123.1; 536/128

(57) ABSTRACT

A method is provided for recovering arabinogalactan from fibrous natural plant material, such as wood from larch trees, said method being a continuous two-step extraction with enrichment method (i.e. counter current extraction) in which preferably water is employed as a solvent. In the counter current process of the invention the fresh solvent initially contacts plant material from which the greater part of the arabinogalactan has been leached and, subsequently, the resulting solution containing the lowest concentration of arabinogalactan contacts the freshest plant material

### FIELD OF THE INVENTION

The present invention relates to a method for recovering arabinogalactan (LAG) from fibrous natural plant materials.

More specifically, the invention relates to a counter current extraction process for recovering arabinogalactan from the wood of trees, such as larch, hemlock, black spruce, douglas fir, cedar, juniper, and sugar maple; especially from the wood of the Larix genus, e.g. Western Larch and Tamarack or Eastern Larch.

### BACKGROUND OF THE INVENTION

Arabinogalactan is of particular commercial interest because its physical properties are well suited for a variety of applications. Arabinogalactan is a polysaccharide, including galactose and arabinose units in varying ratios, which varies in molecular weight from low molecular weight polymers to large macromolecules. Arabinogalactan is completely soluble in water over a wide range of temperatures, and has good emulsification properties. Arabinogalactan remains soluble even at high concentrations, resulting in stable, low viscosity solutions.

Arabinogalactan is used in a wide range of commercial applications, e.g. in the printing, mining, biological research and food industries. For example, in the food industry, arabinogalactan is commonly used as an emulsifier, stabilizer or binder in oils, sweeteners, dressings, flavorings and puddings. Arabinogalactan is also used as a soluble plant-derived dietary fiber.

Methods have been developed for recovering soluble organics, such as arabinogalactan, from fibrous natural plant materials, such as wood. Generally, arabinogalactan is recovered from Larch by chipping or grinding the wood and extracting it with water or dilute acidic solutions. The rate of recovery of arabinogalactan from Larch chips was reported to be dependent upon the extraction temperature and the size of the chip, while the amount of arabinogalactan recovered was dependent on the amount of arabinogalactan in the wood chip. Adams. M. F. and B. V. Ettling. Industrial Gums, 2d ed., R. L. Whistler and J. N. BeMiller, Eds., Academic Press, New York, 1973, p. 415-427.

The state of the art process for extracting arabinogalactan from Larch being employed commercially for large scale production appears to be a water/steam co-current extraction process. The extraction process is designed to remove through pressure the free liquid from the incoming wood chips, re-wet the pressed wood, and reapply pressure. This process is described in FIG. 1. Fresh wood chips are fed to a chip bin where they are heated/moistened with low pressure steam. The steam is absorbed into the wood chips where the heat and condensate work to dissolve the arabinogalactan in the wood chips. Following steam treatment, the chips pass through the first of two plug screw feeders (PSF #1) where the absorbed water is removed via screw pressure and collected in a shared processing vessel. The pressed wood chips transfer to an impregnator which re-soaks the chips in a hot water bath to dissolve the remaining arabinogalactan. Following re-soaking, the chips progress to the second plug screw feeder (PSF #2) where the absorbed water, containing the arabinogalactan, is removed and collected in the same shared processing vessel. The spent pressed wood chips are collected for resale.

The co-current extraction method contains numerous disadvantages. First the co-current method relies on a large amount of water to re-soak the chips which results in a low concentrate arabinogalactan extract when combined with the extract from the first plug screw feeder. Second, the low concentration LAG stream requires a significant amount of energy to remove the excess water. Third, the co-current process generates only moderate yields while leaving significant levels of LAG in the spent wood chip stream. Finally the steam, while necessary to help dissolve the LAG components, also dissolves polyphenols and other color bodies which are believed to create processing issues in the decolorization process for the decolorized LAG products.

U.S. Pat. No. 3,337,526 discloses a process for the extraction of arabinogalactan from finely divided larch wood by passing larch wood chips through an extensive multiple-stage counterflow system. It is the object of this invention to provide an arabinogalactan extract of high concentration and high purity. Thus, the larch wood is extracted or leached in counter current fashion with an aqueous liquid solvent for arabinogalactan. The leaching is so conducted that the liquid solvent containing a low concentration of arabinoglactan contacts wood from which the greater part of the arabinogalacatan has been leached, and the solvent containing a high concentration of arabinogalactan contacts fresh wood. In the preferred embodiment as disclosed in U.S. Pat. No. 3,337,526 fresh solvent initially contacts spent wood and the solvent containing the highest concentration of arabinogalactan contacts the freshest wood. It is stated that arabinogalactan concentrations as high as 35 to 40% are obtained with leaching efficiencies as high as about 90%. In order to achieve these objectives a very complex multiple-stage counterflow process is employed as shown e.g. in FIGS. 1 and 10 of the cited document.

U.S. Pat. No. 5,756,098 discloses methods for the extraction of phytochemicals from fibrous plants (including extraction of arabinogalactan from larch trees) in the absence of solvent.

Arabinogalactan extracts obtained according to the state of the art processes do contain antioxidants and phenolics (e.g. polyphenols) which may have to be removed from the product before further processing it into marketed products such as food supplements for animals and humans or dietary fibers. Phenolics, for example, are one of the main color agents and their presence creates difficulties in a subsequent decolorization process which should lead to a white free flowing powder to be used e.g. as a dietary fiber (FIBERAID®).

### SUMMARY OF ADVANTAGEOUS EMBODIMENTS OF THE INVENTION

It is therefore an object of the present invention to provide a method for efficiently and economically recovering arabinogalactan from natural plant materials while avoiding the disadvantages of the state of the art co-current and counter current extraction processes. It is another object of the present invention to provide a method for recovering arabinogalactan by which, as compared to the known co-current process, the LAG yields are increased while decreasing water consumption at the same time, and by which, as compared to the known counter current processes, a significant reduction of the total antioxidant and phenolic content in the LAG extract can be achieved without employing an extensive multiple-stage counterflow system.

### BRIEF DESCRIPTION OF THE DRAWINGS

FIG. 1 is a schematic representation of an exemplary co-current arabingalactan extraction process; and

FIG. 2 is a schematic representation of an exemplary counter current arabingalactan extraction process in accordance with the invention.

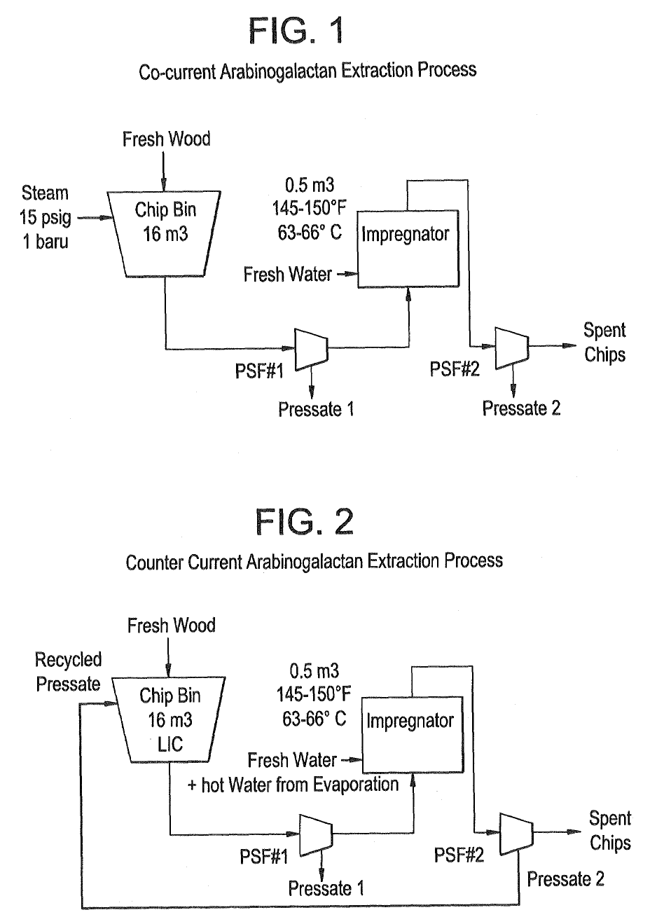

### DETAILED DESCRIPTION OF ADVANTAGEOUS EMBODIMENTS OF THE INVENTION

The method or process for recovering (extracting) arabinogalactan from fibrous natural plant materials is preferably a continuous two-step extraction with enrichment method, which is defined herein as counter current extraction.

A preferred design of the method according to the invention is shown in FIG. 2. Water is used as the preferred solvent for the arabinogalactan (LAG) extraction.

In the counter current process the hot LAG water stream from the second plug screw feeder (PSF #2) is collected in a buffer tank and is recycled to the wood chip stream feeding the chip bin. The chips in the bin are preferably not steamed contrary to the co-current process but are left to soak and absorb the recycled extract. Following the initial soaking and sitting time the warm wet chips pass through the first plug screw feeder (PSF #1) where the absorbed water is removed and collected in a dedicated processing vessel (Pressate 1). The pressed wood chips transfer to an impregnator which re-soaks the chips in a fresh hot water stream to dissolve and extract the remaining arabinogalactan. Following re-soaking, the chips progress to the second plug screw feeder (PSF #2) where the absorbed water, containing the residual arabinogalactan, is removed and collected in the buffer tank where the extract will be recycled into the fresh chip stream (Pressate 2). The spent pressed wood chips are collected for resale.

Thus, the main feature of the method according to the invention is that in a counter current process for producing a solution (preferably aqueous solution) of arabinogalactan from fibrous natural plant materials the fresh solvent initially contacts plant material from which the greater part of the arabinogalactan has been leached/extracted (Impregnator) and that the resulting solvent/solution containing the lowest concentration of arabinogalactan (Pressate 2) contacts the freshest plant material.

The technical equipment described in FIG. 2 which is preferred for the purpose of the present invention can be replaced by other equipment serving the same purpose.

In order to remove absorbed water from plant material a plug screw feeder, applying (screw) pressure, can be replaced by any kind of solid liquid separation equipment such as a filter press or continuous extraction units.

For re-soaking the plant material in a hot solvent (water) stream (Impregnator) any kind of apparatus which brings the solid wood chips in contact with the solvent is suitable such as conveyors or screw-conveyors.

In the following a more detailed description of the invention is given with regard to certain preferred embodiments thereof which can be combined in any manner:

As mentioned above the preferred fibrous natural plant material from which arabinogalactan is leached is the wood of Larch trees (Larix genus), preferably of Western or Eastern Larch trees. The optimum moisture content of the wood as respects water leaching rate and solution concentration is about 20 to 30%, such as about 25%, which corresponds roughly to air dry chips. It is, however, also possible to process fresh wood logs with a higher water content, e.g. about 50 to 70% moisture. The wood is reduced or milled to small pieces so called wood chips. The favourable average chip size is about 1 inch×1 inch×0.2 inch. The leaching rate is directly related to the degree of subdivision of the wood. In the case of chips, it is dependent upon the length of the grain in the chip rather than overall size because diffusion of the sugar from the wood is in the direction of the grain. The smaller the wood particle or the shorter the wood chip in the direction of the grain the higher the leaching rate, or the higher the arabinogalactan concentration of the extract obtainable in a given time at a given temperature. Total leaching time (total residence time) required for a given concentration of arabinogalactan in the leach liquid can easily be ascertained in the laboratory for chips or ground wood of a given size using routine measures.

The process according to the invention is preferably carried out with a liquid solvent, preferably water. The extraction process can be carried out with pure or aqueous mixtures of organic solvents (such as alcohols) or supercritical solvents or their mixtures. The selectivity of the extraction process can be influenced by the addition of salts, acids or bases.

The processing temperature/temperature of the liquid solvent can vary from just above the freezing point of the solution up to about 212° F., and is preferably within the range from about 90 to 150° F. and most preferred from about 90 to 120° F.

The temperature of the fresh solvent/water feed to the impregnator is from about freezing point to 212° F.

The residence time (time for the plant material to soak up and absorb the fresh solvent in the impregnator and to soak up and absorb the recycled extract in the chip bin) is controlled by the PSF speed and is preferably from about 12 to 40 rpms.

The water to used-chip ratio is preferably in the range from about 0.2 to 20 and most preferably about 0.9, leading to an impregnator level of about 50%. The chip bin level is favorably from about 10 to 100% of the total bin volume, especially about 80% of maximum bin volume.

In a preferred embodiment of the invention a pre-soak mixing drum is used to mix the fresh chips with the recycled extract. This conveyor mixes the recycled extract with the fresh chips, provides residence time for soaking and conveys the chips to the chip bin.

The major findings in connection with and advantages of the counter current process according to the invention and as shown by the working examples can be summarized as follows:
The counter current extraction process increases LAG yields by up to about 30% compared with the co-current extraction process at the same dilution levels.
At about the same water consumption the counter current extraction process achieves higher yields of arabinogalactan, typically about 10% to 30% higher compared to co-current extraction. Counter current extraction setup can also be run at same yields as co-current setup, resulting in decreased water consumption by approximately 50% compared to the co-current process.
Implementation of the counter current process alone (vs. co-current process) reduces the total antioxidant and phenolic content in the LAG extract by over 50%.
Implementation of LAG extraction from wood chips containing bark increases the total antioxidant and phenolic extraction compared to standard chips when using the counter current process. The bark addition does have no impact on the molecular weight distribution of the LAG extract stream.
Implementation of direct steam to the chips in the chip bin (as employed in the co-current process) significantly increases the total antioxidant and phenolic levels in the LAG extract and results in a decrease of the molecular weight of the LAG extract.
Combining extraction from chips containing bark and direct steam to the chip bin with the counter current extraction process results in an increase in the total antioxidants and phenolics by about 60% versus the standard co-current process.
Plug Screw Feeder speed and impregnator water level have little impact on the total level of antioxidants and phenolics in the LAG stream. Additionally, neither variable appears to impact on the molecular weight profile of the LAG extract stream.
Residence time is a critical factor as increased time strongly improves the LAG yield in the product.
The temperature of the fresh water feed to the impregnator has no significant impact on the LAG yield.
Increased recycle stream temperature (Pressate 2 in FIG. 2) in principle leads to higher phenolic and antioxidant levels in the LAG extract.
The two step counter current process according to the invention provides a highly efficient and economic method for recovering arabinogalactan from fibrous plant material. The process is easy to handle, can be employed in a batch and continuous mode and can be designed according to the desired product.

The above process features can be combined in any appropriate manner depending on the desired results or product properties. Thus, it can be desirable for example to obtain an LAG extract with a higher phenolic content. Although phenols (compounds containing one or more phenol moieties and include e.g. flavonoids) and other soluble organic compounds are considered impurities with respect to obtaining a refined arabinogalactan solution, these compounds may themselves be desirable as products for other applications either by themselves, or in combination with arabinogalactan. For example, flavonoids have been reported to decrease the amount of putrefactive bacteria while increasing the amount of acid forming bacteria in the human intestine.

The technical equipment used in the extraction process according to the invention, such as plug screw feeder, impregnator, means for controlling the solvent (water) stream and temperature, and the design of such a counter current extraction process in principle, are known to a person with ordinary skill in the art in the pertinent field. Process optimizations and adaptations in order to arrive at the desired products can be made through routine experimentation once the process principle is known. To demonstrate the state of the art knowledge, especially with regard to the above technical features and equipment, the disclosure of U.S. Pat. No. 5,756,098 is hereby incorporated herein by reference in its entirety.

The present invention will be further understood by reference to the following non-limiting examples which are based on the process design as shown in FIG. 2.

### EXAMPLE 1

An initial screening design of experiments (DOE) was performed. The DOE examined the effects of fresh impregnator makeup water temperature and residence time on the LAG and phenolic yield and the LAG concentration in the collected extract.

Variables for the 1st extraction trial:
1. Temperature of Makeup Water (−1/0/+1) 90° F./120° F./150° F.
2. Residence Time in chip bin controlled by PSF Speed (−1/0/+1) 20%/30%/40% motor speeds equal 13 rpm, 19 rpm and 26 rpm actual screw press speeds.

Set-Parameters for 1st extraction trial:
1. Water to Used Chip Ratio ˜0.9 leading to the corresponding Impregnator Level of 50%
2. Chip Bin Level (80% of maximum)
3. Eastern Larch chips ˜1″×1″×⅛″

The DOE design for the trial was a three level full factorial plan with center points to find the optimal conditions. Table I shows the run order of the experimental variables in the trial. Experiments were randomized in order to remove effects from natural LAG content fluctuations.

TABLE I
First Counter Current Extraction Trial Setup

| | | Temperature | | Temperature |
|---|---|---|---|---|
| Run # | PSF Speed % | Deg F. | PSF Speed % | Deg F. |
| 1 | −1 | −1 | 20 | 90 |
| C1 | 0 | 0 | 30 | 120 |
| 2 | 0 | +1 | 30 | 150 |
| 3 | −1 | 0 | 20 | 120 |
| 4 | +1 | +1 | 38 | 150 |
| C2 | 0 | 0 | 30 | 120 |
| 5 | +1 | 0 | 38 | 120 |
| 6 | +1 | −1 | 38 | 90 |
| 7 | −1 | +1 | 20 | 150 |
| C3 | 0 | 0 | 30 | 120 |
| 8 | 0 | −1 | 30 | 90 |

Summary of First Trial Results:
1. The counter current process increases LAG yields by about 30% compared with the co-current extraction process at the same dilution.
2. Counter current extraction achieved higher yields while decreasing water consumption by approximately 50%
3. The counter current extraction process also leads to a significant reduction in the phenolic content of the LAG extract.

The trial was a success in proving the viability of the counter current process in extracting arabinogalactan from the Larch chips. The average LAG yield was about 4.3% (based on total wood weight. Max: 4.9%) for the DOE experiments compared to 3.6% with standard co-current extraction method. Residence time proved to be a critical factor as increased time strongly improved the LAG yield in the product. The assumption is that with co-current setup, the chips are only moistened directly before the initial extraction and, thus, the wet chips have only very short residence times in the chip bin leading to lower yields. With the counter current set-up the extract recycle stream moistens the chips early in the process leading to high residence times. The higher residence times allow the liquid to thoroughly penetrate the wood fibres enhancing the extraction of the LAG. Additionally the higher yields were achieved while decreasing the water required for extraction by approximately 50%. Finally, the temperature of the fresh water feed to the impregnator was not shown to have a significant impact on the LAG yield.

The counter current extraction process overall led to lower phenolic content in the extract stream than standard co-current process. The trials demonstrated that raising the temperature of the fresh water feed to the impregnator slightly increased the phenolic level in the LAG extract. Previous lab work has shown a direct correlation between chip temperature and the level of phenolics in the extract stream. In the co-current process the Larch chips are moistened and heated with steam directly prior to extraction. The counter current process removes the steam and soaks the chips with recycled extract lower in temperature than steam. Based on the lab work and the trial results it is believed that higher chip temperatures in the co-current setup leads to high phenols content.

### EXAMPLE 2

The second counter current extraction trial consisted of three runs: (i) a control run, (ii) a counter current run under optimal conditions and (iii) a counter current run with high temperature extraction to maximize phenol extraction. The control run was the standard co-current extraction operation. Optimal conditions for the counter current run are defined as 30% screw speed, 10 gpm fresh water make-up/recycle rate, & 120 Deg F. make-up water. In the original trial the optimal operating conditions yielded a 6.4% LAG solution. The high temperature run uses the same processing conditions as the optimal counter current design but increases the recycle stream temperature to 160-170° F. The recycle stream temperature was increased by raising the impregnator fresh water feed temperature and adding a steam heat exchanger to the recycle loop from plug screw feeder #2. Previous work from the first counter current extraction trial indicated that increased extraction temperatures lead to higher phenolic and antioxidant levels. All batches were run using the same batch of chips to reduce variation.

Once the LAG extract from the counter current process was collected the material was clarified, cation exchanged, concentrated, decolorized, anion exchanged, carbon treated and reconcentrated. After the first concentration samples of all three runs were taken for ORAC (Oxygen Radical Absorbance Capacity—a measure of antioxidant level) and phenolic testing. Following reconcentration the LAG extract was sent to a Pilot Dryer Facility for drying. The final spray dried product was submitted for limited final product analysis (Carbohydrates, Fat, Protein, & Ash). Since the spray dried product was not agglomerated, physical state, texture, color, bulk density, and particle size testing was not performed. Additionally since the extraction method would have no impact on microbiological testing no analysis was performed.

Summary of Second Trial Results:
1. The counter current extraction process significantly reduced total antioxidants and phenolic content in the LAG extract by over about 50%. Increasing process temperatures only slightly improves the phenol extraction levels.
2. Counter current extraction has no effect on the nutritional properties of the LAG extract.
3. The counter current extraction process leads to no change in the molecular weight profile of the LAG in the final product.

Implementation of the counter current extraction process results in a significant lowering of the antioxidant and phenolic level in the LAG extract as shown by the ORAC and phenolic test results in Table II. When run under optimal conditions the counter current extraction method reduces total antioxidants by about 60% and phenolics by about 56%. Increasing the extraction temperature does improve antioxidant and phenolic extraction. Increasing extraction temperatures resulted in only an about 40% reduction in both total antioxidants and phenolics.

TABLE II
Second Trial ORAC and Phenolic Test Results

| | ORAC total | Phenols Normalized |
|---|---|---|
| | Normalized 50% | 50% |
| Run Conditions | umole TE/g | mg/g |
| Standard Conditions | 179 | 6.18 |
| Optimal Counter Current | 71 | 2.68 |
| High Temp Counter Current | 111 | 3.73 |

Implementation of the counter current extraction process had no impact on the nutritional properties of the product as shown by the test results in Table III. Testing of the samples from the second extraction trial showed no change in the carbohydrates level with the product being well above the >90% specification. Moisture also needs to be subtracted to calculate carbohydrates. Moisture is controlled by the spray drier at ˜4%, so the results in the table need to be reduced by 4%. Additional testing showed no change in the fat, protein and ash levels with the implementation of the counter current extraction process.

TABLE II
Second Trial Nutritional Analysis

| | | | Protein | Ash |
|---|---|---|---|---|
| Run Conditions | Carbohydrates % | Fat % | Vo | n/vo |
| Standard Conditions | 95.56 | 0.12 | 0.31 | 4.01 |
| Optimal Counter Current | 94.57 | 0.07 | 0.35 | 5.01 |
| High Temp Counter Currrent | 96.45 | 0.20 | 0.31 | 3.04 |

What is claimed is:

1. A method for recovering arabinogalactan from fibrous natural plant material by solvent extraction said method comprising the steps of
a) contacting plant material from which the greater part of arabinogalactan has been leached with fresh solvent and
b) contacting the solution obtained in step a) with the freshest plant material.

2. The method of claim 1, wherein the solution obtained in step a) contains the lowest concentration of arabinogalactan.

3. The method of claim 1, wherein the extraction process is carried out with pure or aqueous mixtures of organic solvents or supercritical solvents or their mixtures.

4. The method of claim 3, wherein the organic solvent is an alcohol.

5. The method of claim 1, wherein water is used as the solvent.

6. The method of claim 1, wherein the method is a continuous two or multi-step counter current extraction process.

7. The method of claim 1, wherein the fibrous plant material comprises wood or bark of a plant of the Larix genus.

8. The method of claim 1, wherein the solution obtained in step a) is contacted with the freshest plant material in a presoak mixing drum.

9. The method of claim 1, wherein the optimum moisture content of the fibrous natural plant material is about 20 to 30%.

10. The method of claim 1, wherein the optimum moisture content of the fibrous natural plant material is about 50 to 70%.

11. The method of claim 1, wherein the selectivity of the extraction process is altered by the addition of salts, acids or bases.

12. The method of claim 1, wherein the reaction temperature ranges from about slightly above the freezing point of the solvent up to 212° F.

13. The method of claim 12, wherein the temperature ranges from about 90 to 150° F.

14. The method of claim 12, wherein the temperature ranges from about 90 to 120° F.

15. The method of claim 5, wherein the water to used-plant material ratio is in the range from about 0.2 to 20.

16. The method of claim 5, wherein the water to used plant material ratio is about 0.9.
